(19) United States
(12) Patent Application Publication
(10) Pub. No.: US 2005/0221359 A1
(43) Pub. Date: Oct. 6, 2005

(54) Cospeptin, cosmedin and their uses

(75) Inventors: Aaron J.W. Hsueh, Stanford, CA (US); Sheau Yu Teddy Hsu, Menlo Park, CA (US); Terrance D. Barrett, Encinitas, CA (US); Magda F. Morton, San Diego, CA (US); Clodagh Prendergast, Del Mar, CA (US); Nigel P. Shankley, Solana Beach, CA (US); Orna Avsian-Kretchmer, Fremont, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignees: The Board of Trustees of the Leland Stanford Junior University; Janssen Pharmaceutica N.V.

(21) Appl. No.: 11/088,036

(22) Filed: Mar. 22, 2005

Related U.S. Application Data
(60) Provisional application No. 60/555,818, filed on Mar. 23, 2004.

(51) Int. Cl. C12Q 1/68; C07H 21/04; C12N 15/09; C07K 14/705; A61K 38/17

(52) U.S. Cl. 435/6; 435/69.1; 435/320.1; 435/325; 530/350; 536/23.5; 514/12

(57) ABSTRACT

The invention relates to nucleic acids and polypeptides referred to herein as cospeptin and cosmedin. Cospeptin, cosmedin, analogs and mimetics thereof act in signaling pathways, and are shown to modulate hypertension and other cardiovascular parameters, and gastric emptying. Cospeptin and cosmedin nucleic acid compositions find use in identifying homologous or related proteins and the DNA sequences encoding such proteins; in producing the polypeptide; and in studying associated physiological pathways.

Polypeptide hormones and their receptors play important roles in the maintenance of homeostasis in multicellular organisms. Recent sequencing of the genomes of human and several animal models provide an unprecedented opportunity to identify novel polypeptide ligands based on sequence homology among paralogous ligand genes. In addition, a large number of putative G protein-coupled receptors without known ligands have been predicted based on their characteristic seven transmembrane domains. Although the ligands for some of these ‘orphan’ GPCRs have been identified based on biochemical purification and other approaches, the ligands for many of them are still unknown.

There is considerable interest for clinical and research purposes in the discovery and development of agents that act on these receptors, particularly where there can be enhanced specificity of action over existing ligands.

Related Publications

Methods of discovering new hormones, receptors, and signaling mediators are reviewed by Hsu and Hsueh (2000) Mol Endocrinol 14:594-604. G protein-coupled receptor repertoires are discussed by Vassilatis et al. (2003) P.N.A.S. 100:4903-4908. Orphan G-protein-coupled receptors are discussed by Howard et al. (2001) Trends Pharmacol Sci 22:132-140.

Genetic sequences of interest include Genbank accession number B1756561.1, which is also found in Aceview as Homo sapiens locus ID 80763, a putative secreted or extracellular protein precursor (terori). The mRNA is reported to be 2294 nt long, with 6 exons, ultimately encoding a 116 amino acid polypeptide. The phenotype and in vivo function are reported to be unknown.

Genbank accession number AV702831.1, may be found in Aceview as the H. sapiens soygu locus, also known as 4_{—}25666703. The mRNA is reported to be 1020 nt long, with 3 exons, encoding an 86 amino acid polypeptide predicted to localize in a membrane. No phenotype or in vivo function were reported.

### SUMMARY OF THE INVENTION

Cospeptin and cosmedin nucleic acid compositions and their encoded polypeptides and variants thereof are provided. These peptides are shown to act on cells in the gastrointestinal tract. The peptides also mediate vascular responses for homeostasis, and modulate blood pressure. In one embodiment of the invention, cospeptin and cosmedin peptides find use where it is desirable to modulate blood pressure, to decrease gastric emptying, or to stimulate smooth muscle contraction. Thus, the invention relates to the use of cosmedin and cospeptin and modulators thereof in the treatment of disorders that are benefited from agents useful in delaying and/or slowing gastric emptying. The invention relates to the use of cospeptin or cosmedin modulating agents in the regulation of blood pressure. The invention also relates to the use of cosmedin and cospeptin antagonists to accelerate gastric emptying, for example, in treating gastric hypomotility and associated disorders.

In addition to use as a therapeutic agent, in another embodiment of the invention cospeptin and cosmedin peptides are utilized in screening and research methods for the determination of specific analogs, agonists, antagonists mimetics and agents that modulate their production, metabolism, and disposition. Regulatory peptides are ligands for a subgroup of G protein-coupled receptors (GPCRs) and can play important roles in the gastrointestinal, cardiovascular, hypothalamus-pituitary axis, and the central nervous systems.

In other general embodiments, the invention also provides diagnostics and therapeutics comprising cospeptin and cosmedin nucleic acids, their corresponding genes and gene products, antisense nucleotides, and antibodies specific for one or more epitopes of the cospeptin or cosmedin peptides. The nucleic acid compositions find use in identifying homologous or related genes; for production of the encoded protein; in producing compositions that modulate the expression or function of its encoded protein; for gene therapy; mapping functional regions of the protein; and in studying associated physiological pathways. In addition, modulation of the gene activity in vivo is used for prophylactic and therapeutic purposes.

In one embodiment of the invention, an isolated polypeptide is provided, wherein said polypeptide is encoded by a nucleotide sequence selected from the group consisting of: SEQ ID NO:1; SEQ ID NO:2; a sequence that hybridizes under stringent conditions, as defined herein, to SEQ ID NO:1 or SEQ ID NO:2; and functional fragments, derivatives and homologs thereof. The polypeptide may have an amino acid sequence selected from those set forth in FIGS. 1A and 1B, or at least 6 contiguous residues thereof. Such polypeptides include cosmedin A, B, or C, for example as set forth in SEQ ID NO:3, 4, 5, or a homolog thereof, including, without limitation, the mammalian homologs provided herein. Such polypeptides also include cospeptin A, B, C, D, E, or F, for example as set forth in SEQ ID NO: 6, 7, 8, 9, 10 and 11, or a homolog thereof, including, without limitation, the mammalian homologs provided herein. Such polypeptides may be formulated in a pharmaceutical composition comprising a pharmaceutically acceptable carrier.

In other embodiments of the invention a method of suppressing gastric empyting is provided, wherein a therapeutically effective amount of a cospeptin or cosmedin peptide is administered. Such administering may be performed on an individual suffering from a gastrointestinal disorder such as spasm, post-prandial dumping syndrome, post-prandial hyperglycemia, etc.; prior to a gastrointestinal diagnostic procedure; and the like.

In yet other embodiments of the invention, a method of regulating gastric empyting is provided, wherein a therapeutically effective amount of an antagonist/agonist of cosmedin or cospeptin is administered. Such administering may be performed on an individual suffering from, for example, diabetic neuropathy; anorexia nervosa; and the like.

Other aspects of the invention and their features and advantages will become apparent from the following description and the appended claims.

### BRIEF DESCRIPTION OF THE DRAWINGS

FIG. 1A. Human cosmedin sequence. FIG. 1B. Comparison of cosmedin from diverse vertebrate species.

FIG. 2A. Human cospeptin sequence. FIG. 2B. Comparison of cosmedin from diverse vertebrate species,

FIG. 3A. RT-PCR analyses of the tissue expression pattern of cosmedin in human. FIG. 3B. RT-PCR analyses of the tissue expression pattern of cosmedin in mouse. FIG. 3C. Cosmedin expression in human GI tissues.

FIG. 4A. RT-PCR analyses of the tissue expression pattern of cospeptin in human tissues. FIG. 4B. RT-PCR analyses of the tissue expression pattern of cospeptin in human GI tissues.

FIG. 5A. Biological actions of cosmedin-B peptide, gastric emptying assay. FIG. 5B. Biological actions of cospeptin peptides. FIG. 5C. Actions of Cosmedin-B in rat ileum contractility assay.

FIG. 6A. Regulation of blood pressure by cospeptin. FIG. 6B. regulation of heart rate by cospeptin.

FIG. 7. Saturation curve for Tyr0-cospeptin E binding to the rat pituitary gland showing a Kd value of 5 nM.

FIG. 8. Binding of Tyr0-cospeptin to diverse rat tissues showing high binding sites in pituitary and ovary, and lower binding sites in testis, lung, duodenum, hypothalamus, and heart.

FIG. 9 Hormonal specificity of Tyr0-cospeptin binding to its receptors in the pituitary. Competition studies indicated the following order of affinity to the cospeptin receptors: cospeptin E=Tyr0-cospeptin E>cospeptin A,B>>cospeptin F>>cosmedin.

### DETAILED DESCRIPTION OF THE INVENTION AND PREFERRED EMBODIMENTS

The regulatory peptides cosmedin and cospeptin are ligands for a subgroup of G protein-coupled receptors (GPCRs) that play important roles in gastrointestinal, cardiovascular, hypothalamus-pituitary axis, and the central nervous system. In addition to use as a therapeutic agent, cospeptin and cosmedin peptides are utilized in screening and research methods for the determination of specific analogs, agonists, antagonists and mimetics and inhibitors of their production, metabolism and disposition.

In one embodiment of the invention, modulators of cospeptin or cosmedin activity are used in the treatment of inflammatory bowel disease. In another embodiment of the invention, modulators of cospeptin or cosmedin activity are used in the treatment of gastric or intestinal hypersecretion; gastric atony, urinary retention, reflux esophagitis, motion sickness, anorexia nervosa, nausea and vomiting, e.g. due to chemotherapy, diabetic gastropariesis, etc.

In another embodiment, modulators of cospeptin or cosmedin activity are used in the control of cardiovascular parameters, including hypertension.

Modulators of cospeptin or cosmedin activity refer to molecules that alter the physiological function of cospeptin or cosmedin, including, without limitation, the production, metabolism, disposition of the peptide, etc. Such modulators include agonists, which enhance, potentiate and/or mimic the activity of a cospeptin or cosmedin peptide; and antagonists, which inhibit or decrease the activity of a cospeptin or cosmedin peptide.

In one aspect, the invention features a method of beneficially regulating gastrointestinal motility in a subject by administering to said subject a therapeutically effective amount of an cospeptin or cosmedin or modulator thereof. In one embodiment, the methods of the present invention are directed to reducing gastric motility. In another embodiment, the invention is directed to methods of delaying gastric emptying. These methods may be used on a subject undergoing a gastrointestinal diagnostic procedure, for example radiological examination or magnetic resonance imaging. Alternatively, these methods may be used to reduce gastric motility in a subject suffering from a gastrointestinal disorder, for example, spasm (which may be associated with acute diverticulitis, a disorder of the biliary tract or a disorder of the Sphincter of Oddi). In another aspect, the invention is directed to a method of treating post-prandial dumping syndrome in a subject by administering to the subject a therapeutically effective amount of a cospeptin or cosmedin modulator. In another aspect, the invention is directed to a method of treating post-prandial hyperglycemia by administering to a subject a therapeutically effective amount of a cospeptin or cosmedin agonist, e.g. post-prandial hyperglycemia as a consequence of Type 2 diabetes mellitus.

In another aspect, the present invention is directed to a method of treating gastric hypomotility in a subject by administering to the subject a therapeutically effective amount of a cospeptin or cosmedin antagonist. These methods may be employed where hypomotility is a consequence of diabetic neuropathy or where hypomotility is a consequence of anorexia nervosa. Hypomotility may also occur as a consequence of achlorhydria or as a consequence of gastric surgery. In another aspect, the invention is directed to a method of accelerating gastric emptying in a subject by administering to the subject a therapeutically effective amount of a cospeptin or cosmedin modulator.

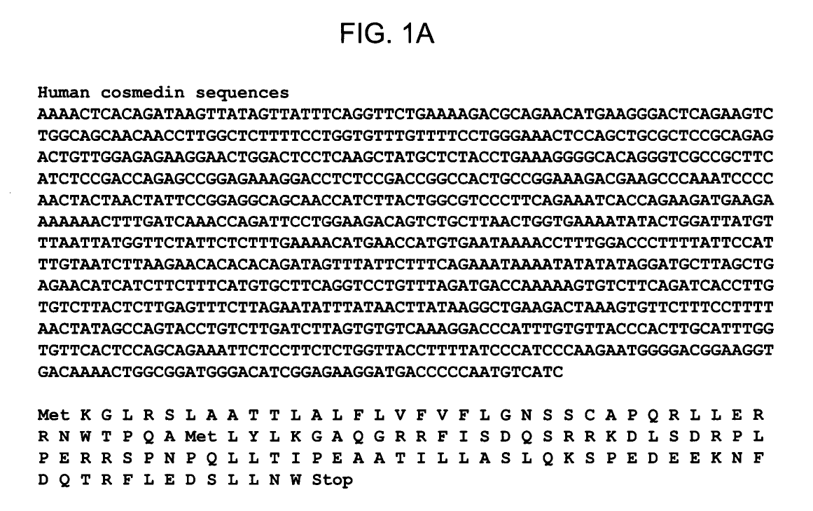

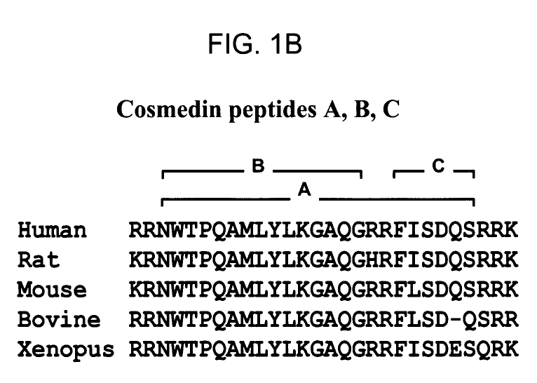

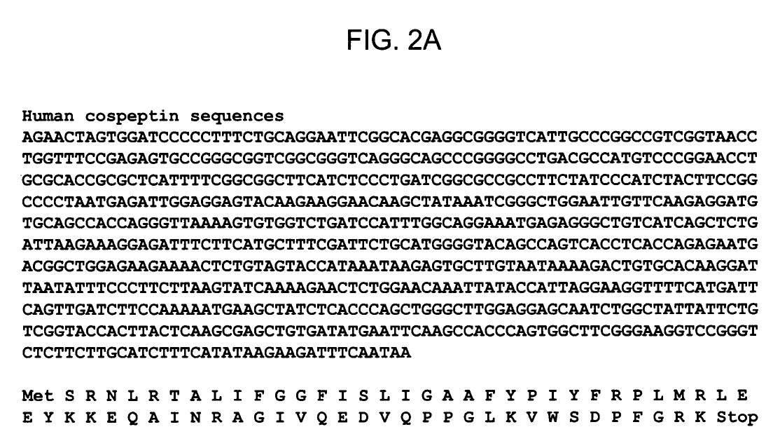

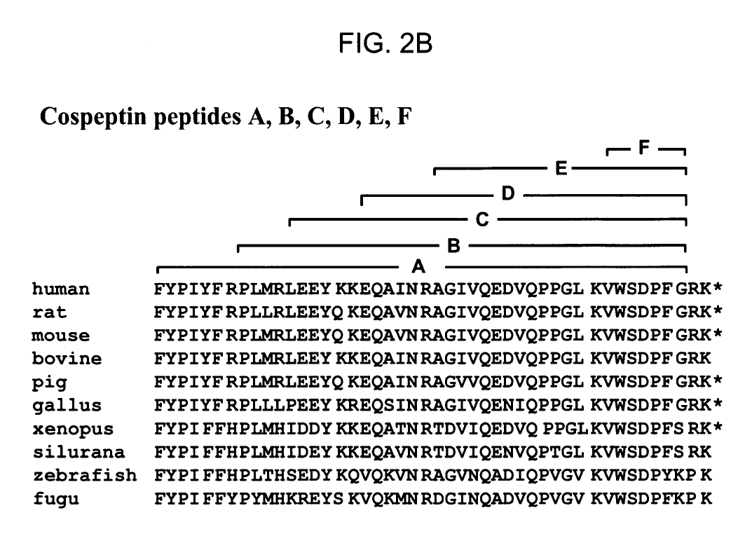

The invention provides nucleic acids and polypeptides referred to herein as cospeptin and cosmedin. Forms of cosmedin include cosmedin A, cosmedin B, and cosmedin C, which are 23, 15, and 6 amino acids in length, as shown in FIG. 1B. Forms of cospeptin include cospeptin A, cospeptin B, cospeptin C, cospeptin D, and cospeptin E, cospeptin F which are 43, 37, 33, 27, 21, and 7 amino acid residues in length, as shown in FIG. 2B. The peptides are shown herein to be biologically active, including effects on cells in the gastrointestinal tract.

The cosmedin gene, provided as SEQ ID NO:1, encodes a propeptide of 116 amino acids in length (as shown in FIG. 1A), which is processed to the mature forms A, B, C. In humans, the mature forms of cosmedin may comprise:

| | | |
|---|---|---|
| Form A,, | SEQ ID NO: 3 | NWTPQAMLYLKGAQGRRFISDQS |
| Form B,, | SEQ ID NO: 4 | NWTPQAMLYLKGAQG |
| Form C,, | SEQ ID NO: 5 | FISDQS. |

The peptides may be provided as a free acid at the carboxy terminus, or may be amidated. The conserved glycine residue indicates that cosmedin B is naturally amidated at the conserved terminal glycine residue. Cosmedin transcripts are expressed in diverse human gastrointestinal tract tissues, as well as hypothalamus, pituitary, kidney, pancreas, and other tissues. The form cosmedin B is particularly active, producing a concentration dependent muscle contraction of guinea-pig ileum comparable to that of the acetylcholine muscarinic receptor agonist 5-methylfurmethide. Administration of cosmedin B in vivo effectively inhibits gastric emptying.

The cospeptin gene, provided as SEQ ID NO:2, encodes a propeptide of 67 amino acids in length (as shown in FIG. 2A), which is processed to the mature forms A, B, C, D, E, and F. In humans, the mature forms of cospeptin may comprise:

Form A,,
SEQ ID NO: 6
FYPIYFRPLMRLEEYKKEQAINRAGIVQEDVQPPGLKVWSDPFG
Form B,,
SEQ ID NO: 7
PLMRLEEYKKEQAINRAGIVQEDVQPPGLKVWSDPFG
Form C,,
SEQ ID NO: 8
LEEYKKEQAINRAGIVQEDVQPPGLKVWSDPFG
Form D,,
SEQ ID NO: 9
EQAINRAGIVQEDVQPPGLKVWSDPFG
Form E,,
SEQ ID NO: 10
AGIVQEDVQPPGLKVWSDPFG
Form F,,
SEQ ID NO: 11
VWSDPFG.

The peptides may be provided as a free acid at the carboxy terminus, or may be amidated. The conserved glycine residue indicates that all six forms may be naturally amidated at the conserved terminal glycine residue. Cospeptin transcripts are expressed in diverse human gastrointestinal tract tissues, as well as hypothalamus, pancreas, thymus, and other tissues. Treatment with cospeptin-A, -B, and -E, effectively inhibits gastric emptying.

For use in the subject methods, any of the native cospeptin or cosmedin forms, including peptides comprising the amino acid sequence set forth according to any of SEQ ID NO:3-11 and homologs thereof, modifications thereof, or a combination of forms may be used. Peptides of interest include fragments of at least about 6 contiguous amino acids, more preferably at least about 10 contiguous amino acids, and may comprise a total of 15 or more amino acids, up to the provided peptide sequences, and may extend further to comprise other sequences present in the precursor protein.

The sequence of the cospeptin or cosmedin peptides may be altered from those provided herein in various ways known in the art to generate targeted changes in sequence. The altered peptide will be substantially similar to the sequences provided herein, and preferably will differ by from one to three amino acids. The sequence changes may be suitable substitutions, insertions or deletions. These modified peptides may be used as modulators of cosmedin/cospeptin activity.

The cospeptin or cosmedin peptides may be joined to a variety of other oligopeptides or proteins for a variety of purposes. Various post-translational modifications may be achieved. For example, by employing the appropriate coding sequences, one may provide farnesylation or prenylation. In this situation, the peptide will be bound to a lipid group at a terminus, so as to be able to be bound to a lipid membrane, such as a liposome.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. Preferably, the compositions which are used will comprise at least 20% by weight of the desired product, more preferably at least about 75% by weight, even more preferably at least about 95% by weight, and for therapeutic purposes, preferably at least about 99.5% by weight, in relation to contaminants related to the method of preparation of the product and its purification. The percentages are based upon total protein.

In one preferred embodiment of the invention, the therapeutic peptide consists essentially of a polypeptide sequence set forth herein. By “consisting essentially of” in the context of a polypeptide described herein, it is meant that the polypeptide is composed of a sequence, e.g. a sequence set forth in the seqlist, which sequence may be flanked by one or more amino acid or other residues that do not materially affect any basic characteristics of the polypeptide.

### COMPOUND SCREENING

In another aspect, the invention relates to methods for assaying or screening compounds to determine their activities as modulators of the function of the polypeptides described above. Compound screening may be performed using an in vitro model, a genetically altered cell or animal, or purified protein corresponding to any one of the cospeptin or cosmedin forms. One can identify ligands or substrates that bind to, modulate or mimic the action of the peptides, including the identification of modulators.

The polypeptides include those provided herein, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain where the polypeptide is a member of a protein family, or a region associated with a consensus sequence). Variants also include fragments of the polypeptides disclosed herein, for example, biologically active fragments and/or fragments corresponding to functional domains.

Compound screening identifies modulating agents that modulate function of cospeptin or cosmedin. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like.

The term “modulator” includes any molecule, e.g. protein or pharmaceutical, with the capability of altering or mimicking the physiological function of a cospeptin or cosmedin peptide. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate modulators comprise functional groups necessary for structural interaction with proteins, particularly hydrogen bonding, and typically include at least an amine, carbonyl, hydroxyl or carboxyl group, preferably at least two of the functional chemical groups. The candidate modulator often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate modulators are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to, or interfering in the binding of cospeptin or cosmedin. The binding assays usually involve contacting cospeptin or cosmedin with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots, etc.

The level of expression or activity can be compared to a baseline value. The baseline value can be a value for a control sample or a statistical value that is representative of a control population. Expression or activity levels can also be determined for cells that do not respond to cospeptin or cosmedin as a negative control.

Compounds that are initially identified by any of the foregoing screening methods can be further tested to validate the apparent activity. The basic format of such methods involves administering a lead compound identified during an initial screen to an animal that serves as a model for humans and then determining whether the desired biological function is affected. The animal models utilized in validation studies generally are mammals. Specific examples of suitable animals include, but are not limited to, primates, mice, and rats.

Active test agents identified by the screening methods described herein that modulate cospeptin or cosmedin activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate cospeptin or cosmedin activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

### PHARMACEUTICAL COMPOSITIONS

Active compounds identified by the screening methods described above and analogs thereof (e.g., pharmaceutically acceptable salts) can serve as the active ingredient in pharmaceutical compositions formulated for the treatment of various disorders as described above. The active ingredient is present in a therapeutically effective amount, i.e., an amount sufficient when administered to substantially modulate the effect of the targeted protein or polypeptide to treat a disease or medical condition mediated thereby.

The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

When the pharmaceutical composition includes a polypeptide as the active ingredient, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which are composed of the packaged active ingredient with a suppository base. Suitable suppository bases include natural or synthetic triglycerides or paraffin hydrocarbons. In addition, it is also possible to use gelatin rectal capsules, which are composed of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### ANTIBODIES SPECIFIC FOR COSPEPTIN OR COSMEDIN POLYPEPTIDES

The present invention further provides antibodies specific for cospeptin or cosmedin polypeptides, e.g. any one of the variants, polypeptides, or domains described above. Such antibodies are useful, for example, in methods of detecting the presence of cospeptin or cosmedin in a biological sample, and in methods of isolating cospeptin or cosmedin from a biological sample. Antibodies may also be useful as antagonists of cospeptin or cosmedin activity.

The cospeptin or cosmedin polypeptides of the invention are useful for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme that generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a cospeptin or cosmedin polypeptide, particularly a human cospeptin or cosmedin polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For a more detailed description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### USES OF COSPEPTIN OR COSMEDIN

In light of the pharmacologic activities of cospeptin and cosmedin, numerous clinical indications are evident. For example, clinical indications for which a cospeptin or cosmedin peptide modulator may find use include the treatment of inflammatory bowel disease. In another embodiment of the invention, modulators of cospeptin or cosmedin activity are used in the treatment of gastric or intestinal hypersecretion; gastric atony, urinary retention, reflux esophagitis, motion sickness, anorexia nervosa, nausea and vomiting, e.g. due to chemotherapy, diabetic gastropariesis, etc.

Irritable bowel syndrome (IBS), a chronic or recurring gastrointestinal disorder, produces abdominal pain or discomfort in its victims. IBS presents itself as abdominal pain accompanied by altered bowel habits. At present, treatment options range from education and dietary modification to drug therapy to psychological therapy. Drug and/or psychological therapy is called for in those 30% of IBS patients with moderate or severe symptoms. The symptoms of IBS are a product of quantitative differences in the motor reactivity of the intestinal tract, and increased sensitivity to stimuli or spontaneous contractions. A hallmark of IBS is abdominal pain that is relieved by defecation, and which is associated with a change in the consistency or frequency of stools. IBS may be diarrhea-predominant, constipation-predominant, or an alternating combination of both.

Persons with IBS exhibit hypersensitivity, particularly hyperalgesia, in response to painful distensions in the small bowel and colon and to normal intestinal function. Furthermore, there are also increased or unusual areas of visceral pain. The abdominal pain is often poorly localized, and may be migratory and/or variable in nature. The pain may be worsened by meals and reduced upon defecation. Furthermore, IBS symptoms, including hyperalgesia, are commonly initiated or exacerbated by stress.

Antispasmodic medication may be prescribed for IBS pain and bloating. Activities of agents effective for relieving irritable bowel syndrome symptoms are generally spasmolytic, which thereby decrease intestinal motility.

Gastric hypomotility with delayed emptying of liquid and/or solid contents is a component of a number of gastrointestinal disorders, and may be treated with modulators of cospeptin or cosmedin. For a general discussion, see Goodman and Gilman's The Pharmacological Basis of Therapeutics, Chapter 38 (Pergamon Press, Eighth Edition 1990). The symptoms of such disorders may include nausea, vomiting, heartburn, postprandial discomfort, and indigestion. Gastroesophageal reflux is often evident and can give rise to esophageal ulceration; there may also be respiratory symptoms or intense substernal pain that can be confused with asthma or myocardial infarction, respectively. Although the cause is unknown in the majority of patients, gastric stasis or hypomotility is frequently a consequence of diabetic neuropathy; this condition is also often present in patients with anorexia nervosa or achlorhydria or following gastric surgery.

The medical management of patients with gastric hypomotility usually includes the administration of a prokinetic agent. Although antiemetic phenothiazines or bethanechol may provide some relief, these drugs do not accelerate gastric emptying in the vast majority of patients and often produce unacceptable side effects.

Agents that serve to delay gastric emptying have found a place in medicine as well, particularly as diagnostic aids in gastro-intestinal radiologic examinations. Such agents are also used to treat various painful gastrointestinal disorders associated with spasm. The cospeptin and cosmedin peptides described above are useful in view of their pharmacological properties to regulate emptying.

Clinical indications for which a cospeptin or cosmedin peptide modulator may find use also include the treatment of hypertension. Hypertension is a disease, which if untreated, strongly predisposes to atherosclerotic cardiovascular disease. It is estimated that as many as 1 in 4 adult Americans have hypertension. Hypertension is approximately twice as common in persons with diabetes as in those without. The prevalence of hypertension increases with age.

Hypertension should not be diagnosed on the basis of a single measurement. Initial elevated readings should be confirmed on at least two subsequent visits over one week or more with average diastolic blood pressure of 90 mmHg or greater or systolic blood pressure of 140 mmHg or greater required for diagnosis of hypertension. Special care is warranted in diagnosing hypertension in persons with diabetes because of greater variability of blood pressure and a much greater likelihood of isolated systolic hypertension. A goal blood pressure of less than 130/85 mmHg is recommended for these patients.

In addition to dietary changes, pharmacological treatment may be required to control high blood pressure. The subject peptides may be administered to reduce arterial blood pressure. In addition, a secondary effect of reducing hypertension is reduction of edema and inflammatory exudate volume.

### COSPEPTIN AND COSMEDIN NUCLEIC ACIDS

The invention includes nucleic acids having a sequence set forth in SEQ ID NO:1 and SEQ ID NO:2; nucleic acids that hybridize under stringent conditions, preferably conditions of high stringency, to the sequences set forth in SEQ ID NO:1 and SEQ ID NO:2; genes corresponding to the provided nucleic acids; sequences encoding cospeptins; and functional fragments and derivatives thereof. Other nucleic acid compositions contemplated by and within the scope of the present invention will be readily apparent to one of ordinary skill in the art when provided with the disclosure here.

The nucleic acids of the invention include nucleic acids having sequence similarity or sequence identity to SEQ ID NO:1 and SEQ ID NO:2. Nucleic acids having sequence similarity may be detected by hybridization under low stringency conditions, for example, at 50° C. and 10×SSC (0.9 M saline/0.09 M sodium citrate) and remain bound when subjected to washing at 55° C. in 1×SSC. More preferably, sequence identity is determined by hybridization under high stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to SEQ ID NO:1 or SEQ ID NO:2 under stringent hybridization conditions. By using probes, particularly labeled probes of the DNA sequences, one can isolate homologous or related genes. The source of homologous genes can be any species, e.g. primate species, preferably human, rodents, such as rats and mice; canines, felines, bovines, ovines, equines, fish, yeast, nematodes.

In one embodiment, hybridization is performed using at least 18 contiguous nucleotides (nt) of SEQ ID NO:1 and SEQ ID NO:2, or a DNA encoding any one of the provided peptides set forth in FIGS. 1B and 2B. Such a probe will preferentially hybridize with a nucleic acid comprising the complementary sequence, allowing the identification and retrieval of the nucleic acids that uniquely hybridize to the selected probe. Probes of more than 18 nt can be used, e.g., probes of from about 18 nt to about 25, 50, 100, 250, or 500 nt, but 18 nt usually represents sufficient sequence for unique identification.

Nucleic acids of the invention also include naturally occurring variants of the nucleotide sequences (e.g., degenerate variants, allelic variants). Variants of the nucleic acids of the invention are identified by hybridization of putative variants with nucleotide sequences disclosed herein, preferably by hybridization under stringent conditions. For example, by using appropriate wash conditions, variants of the nucleic acids of the invention can be identified where the allelic variant exhibits at most about 25-30% base pair (bp) mismatches relative to the selected nucleic acid probe. In general, allelic variants contain 15-25% bp mismatches, and can contain as little as even 5-15%, or 2-5%, or 1-2% bp mismatches, as well as a single bp mismatch.

The invention also encompasses homologs corresponding to the nucleic acids of SEQ ID NO:1 and SEQ ID NO:2, or a DNA encoding any one of the provided peptides set forth in FIGS. 1B and 2B., where the source of homologous genes can be any mammalian species, e.g., primate species, particularly human; rodents, such as rats; canines, felines, bovines, ovines, equines, fish, yeast, and nematodes. Between mammalian species, e.g., human and mouse, homologs generally have substantial sequence similarity, e.g., at least 75% sequence identity, preferably at least 90%, and more preferably at least 95% between nucleotide sequences. Sequence similarity is calculated based on a reference sequence, which may be a subset of a larger sequence, such as a conserved motif, coding region, or flanking region. A reference sequence will preferably be at least about 18 contiguous nt long, more preferably at least about 30 nt long, and may extend to the complete sequence that is being compared. Algorithms for sequence analysis are known in the art, such as gapped BLAST, described in Altschul et al. Nucl. Acids Res. (1997) 25:3389-3402.

The subject nucleic acids can be cDNAs or genomic DNAs, as well as fragments thereof, preferably fragments that encode a biologically active polypeptide and/or are useful in the methods disclosed herein (e.g., in diagnosis, or as a unique identifier of a differentially expressed gene of interest). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters and enhancers, including about 1 kb, but possibly more, of flanking genomic DNA at either the 5′ and 3′ end of the transcribed region. The genomic DNA can be isolated as a fragment of 100 kbp or smaller and is substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject polypeptides. Double or single stranded fragments can be obtained from the DNA sequence by chemically synthesizing oligonucleotides in accordance with conventional methods, by restriction enzyme digestion, by PCR amplification, and the like. Isolated nucleic acids and nucleic acid fragments of the invention comprise from at least about 18, preferably about 50, more preferably about 100, to about 500 contiguous nt selected from the nucleic acid sequence as shown in SEQ ID NO:1 and SEQ ID NO:2. Preferably, fragments will be of at least 18 nt, more preferably at least 25 nt or up to about 50 contiguous nt in length or more.

Probes specific to the nucleic acid of the invention can be generated using the nucleic acid sequence disclosed in SEQ ID NO:1 and SEQ ID NO:2, or a DNA encoding any one of the provided peptides set forth in FIGS. 1B and 2B. The probes are preferably at least about 18 nt, 25 nt or more of the corresponding contiguous sequence. The probes can be synthesized chemically or can be generated from longer nucleic acids using restriction enzymes. The probes can be labeled, for example, with a radioactive, biotinylated, or fluorescent tag. Preferably, probes are designed based upon an identifying sequence of one of the provided sequences. More preferably, probes are designed based on a contiguous sequence of one of the subject nucleic acids that remain unmasked following application of a masking program for masking low complexity (e.g., BLASTX) to the sequence, i.e., one would select an unmasked region, as indicated by the nucleic acids outside the poly-n stretches of the masked sequence produced by the masking program.

The nucleic acids of the subject invention are isolated and obtained in substantial purity, generally as other than an intact chromosome. Preferably, the nucleic acids, either as DNA or RNA, will be obtained substantially free of other naturally-occurring nucleic acid sequences, generally being at least about 50%, preferably at least about 90% pure and are preferably “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

### MODULATION OF COSPEPTIN OR COSMEDIN EXPRESSION

The cospeptin or cosmedin genes, gene fragments, or the encoded protein or protein fragments are useful in gene therapy to treat disorders associated with cospeptin or cosmedin defects. Antisense cospeptin sequences may be administered to inhibit expression. Other inhibitors or modulators are identified by screening for biological activity in a cospeptin- or cosmedin-based binding assay.

Expression vectors may be used to introduce the cospeptin or cosmedin gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more preferably for a period of at least about several days to several weeks.

The gene or cospeptin or cosmedin peptide may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the cospeptin or DNA, then bombarded into skin cells.

Antisense molecules can be used to down-regulate expression of cospeptin or cosmedin in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, preferably at least about 12, more preferably at least about 20 nucleotides in length, and not more than about 500, preferably not more than about 50, more preferably not more than about 35 nucleotides in length, where the length is governed by efficiency of inhibition, specificity, including absence of cross-reactivity, and the like.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Agents that block cospeptin and/or cosmedin activity provide a point of intervention in an important signaling pathway. Numerous agents are useful in reducing cospeptin and/or cosmedin activity, including agents that directly modulate cospeptin or cosmedin expression as described above, e.g. expression vectors, anti-sense specific for cospeptin or cosmedin; and agents that act on the cospeptin or cosmedin protein, e.g. cospeptin or cosmedin specific antibodies and analogs thereof, and small organic molecules that block cospeptin or cosmedin binding activity

### DIAGNOSTIC USES

DNA-based reagents derived from the sequence of cospeptin or cosmedin, e.g. PCR primers, oligonucleotide or cDNA probes, as well as antibodies against cospeptin or cosmedin, are used to screen patient samples, e.g. biopsy-derived tissues, blood samples, and the like, for amplified cospeptin or cosmedin DNA, or increased expression of cospeptin or cosmedin mRNA or proteins. DNA-based reagents are also designed for evaluation of chromosomal loci implicated in certain diseases e.g. for use in loss-of-heterozygosity (LOH) studies, or design of primers based on cospeptin or cosmedin coding sequence.

The polynucleotides of the invention can be used to detect differences in expression levels between two samples. A difference between the protein levels, or the mRNA in the two tissues that are compared, for example, in molecular weight, amino acid or nucleotide sequence, or relative abundance, indicates a change in the gene, or a gene, which regulates it, in the tissue of the human that was suspected of being diseased.

The subject nucleic acid and/or polypeptide compositions may be used to analyze a patient sample for the presence of polymorphisms associated with a disease state or genetic predisposition to a disease state. Biochemical studies may be performed to determine whether a sequence polymorphism in a cospeptin and/or cosmedin coding region or control regions is associated with disease, such as stress related disorders, e.g. anxiety disorders. Disease associated polymorphisms may include deletion or truncation of the gene, mutations that alter expression level, that affect the binding activity of the protein, the kinase activity domain, and the like.

Changes in the promoter or enhancer sequence that may affect expression levels of cospeptin and/or cosmedin can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, and chloramphenicol acetyltransferase which provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express cospeptin or cosmedin may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook et al., Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, and ³H. The label may be a two stage system, where the amplified DNA is conjugated to biotin, haptens, and the like having a high affinity binding partner, e.g. avidin and specific antibodies., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g., amplified or cloned fragment, is analyzed by one of a number of methods known in the art. The nucleic acid may be sequenced by dideoxy or other methods, and the sequence of bases compared to a wild-type cospeptin or cosmedin sequence. Hybridization with the variant sequence may also be used to determine its presence, by Southern blots, dot blots, and the like. The hybridization pattern of a control and variant sequence to an array of oligonucleotide probes immobilized on an array may also be used as a means of detecting the presence of variant sequences. Single strand conformational polymorphism (SSCP) analysis, denaturing gradient gel electrophoresis (DGGE), and heteroduplex analysis in gel matrices may be used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, preferably acrylamide or agarose gels.

Screening for mutations in cospeptin and/or cosmedin may be based on the functional or antigenic characteristics of the protein. Protein truncation assays are useful in detecting deletions that may affect the biological activity of the protein. Various immunoassays designed to detect polymorphisms in cospeptin and/or cosmedin proteins may be used in screening. Where many diverse genetic mutations lead to a particular disease phenotype, functional protein assays have proven to be effective screening tools. The activity of the encoded cospeptin and/or cosmedin protein in binding assays may be determined by comparison with the wild-type protein. Proteins may also be screened for the presence of post-translational modification of the cospeptin or cosmedin proteins, e.g. under pathological conditions, including proteolytic fragments, amidation, and acetylation.

Antibodies specific for cospeptin or cosmedin may be used in staining or in immunoassays. Samples, as used herein, include biological fluids such as blood, cerebrospinal fluid, dialysis fluid and the like; organ or tissue culture derived fluids; and fluids extracted from physiological tissues. Also included are derivatives and fractions of such fluids. The cells may be dissociated, in the case of solid tissues, or tissue sections may be analyzed. Alternatively a lysate of the cells may be prepared.

Diagnosis may be performed by a number of methods to determine the absence or presence or altered amounts of normal or abnormal cospeptin or cosmedin in patient cells. For example, detection may utilize staining of cells or histological sections, performed in accordance with conventional methods. Cells are permeabilized to stain cytoplasmic molecules. The antibodies of interest are added to the cell sample, and incubated for a period of time sufficient to allow binding to the epitope, preferably at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein rhodamine, Texas red, and the like. Final detection uses a substrate that undergoes a color change in the presence of the peroxidase. The absence or presence of antibody binding may be determined by various methods, including flow cytometry of dissociated cells, microscopy, radiography, scintillation and counting.

In some embodiments, the methods are adapted for use in vivo. In these embodiments, a detectably-labeled moiety, e.g., an antibody, which is specific for cospeptin or cosmedin is administered to an individual (e.g., by injection), and labeled cells are located using standard imaging techniques, including, but not limited to, magnetic resonance imaging, computed tomography scanning, and the like.

Diagnostic screening may also be performed for polymorphisms that are genetically linked to a disease predisposition, preferably through the use of microsatellite markers or single nucleotide polymorphisms. The microsatellite polymorphism itself is in many cases not phenotypically expressed, but is linked to sequences that result in a disease predisposition. However, in some cases the microsatellite sequence itself may affect gene expression. Microsatellite linkage analysis may be performed alone, or in combination with direct detection of polymorphisms, as described above. The use of microsatellite markers for genotyping is well known. For examples, see Mansfield et al. (1994) Genomics 24:225-233; Ziegle et al. (1992) Genomics 14:1026-1031; Dib et al., supra.

The detection methods can be provided as part of a kit. Thus, the invention further provides kits for detecting the presence of an mRNA encoding cospeptin or cosmedin, and/or a polypeptide encoded thereby, in a biological sample. Procedures using these kits may be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

### GENETICALLY ALTERED CELL OR ANIMAL MODELS FOR COSPEPTIN AND COSMEDIN FUNCTION

The subject nucleic acids can be used to generate transgenic animals or site specific gene modifications in cell lines. Transgenic animals may be made through homologous recombination, where the normal cospeptin or cosmedin locus is altered. Alternatively, a nucleic acid construct is randomly integrated into the genome. Vectors for stable integration include plasmids, retroviruses and other animal viruses, YACs, and the like.

The modified cells or animals are useful in the study of cospeptin or cosmedin function and regulation. For example, a series of small deletions and/or substitutions may be made in the cospeptin gene to determine the role of different residues in receptor binding or signal transduction. In one embodiment, cospeptin or cosmedin is used to construct transgenic animal models for disorders where expression of cospeptin is specifically altered, i.e. reduced, increased, or absent. Specific preferred constructs include anti-sense cospeptin or cosmedin, which will block cospeptin and/or cosmedin expression and expression of dominant negative cospeptin or cosmedin mutations. A detectable marker, such as lac Z, may be introduced into the cospeptin or cosmedin locus, where up-regulation of cospeptin or cosmedin expression will result in an easily detected change in phenotype.

One may also provide for expression of the cospeptin or cosmedin gene or variants thereof in cells or tissues where it is not normally expressed or at abnormal times of development. By providing expression of cospeptin or cosmedin protein in cells in which it is not normally produced, one can induce changes in cell behavior, e.g. in the control of cell growth and tumorigenesis.

DNA constructs for homologous recombination will comprise at least a portion of the cospeptin and/or cosmedin gene with the desired genetic modification, and will include regions of homology to the target locus. The regions of homology may include coding regions, or may utilize intron and/or genomic sequence. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line may be employed, or embryonic cells may be obtained freshly from a host, e.g. mouse, rat, or guinea pig. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in culture. The transgenic animals may be any non-human mammal, such as laboratory animals and domestic animals. The transgenic animals may be used in functional studies, drug screening, and the like to determine the effect of a candidate drug on stress responses.

### EXPERIMENTAL

The following examples are put forth for illustrative purposes, and are not intended to limit the scope of what the inventors regard as their invention. Unless indicated otherwise, parts are parts by weight, molecular weight is weight average molecular weight, temperature is in degrees Celsius, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. Accordingly, it should be understood that the scope of the invention is not limited by this detailed description, but by the appended claims as properly construed under principles of patent law.

### EXAMPLE 1

Methods and Materials

Bioinformatic analyses A database was established by sorting and entering all proteins in the human proteome with a signal peptide for secretion but without a transmembrane domain. Candidate peptide hormones were identified using a regular expression search for putative peptides between 8 and 50 residues in length with flanking dibasic proteolytic cleavage sites: (([KR]{2})([A-Z]{8,50})([KR]{2})([A-Z]{0,50})$). The presumed mature peptide region was used to query genomes of diverse vertebrates before confirmation of the evolutionary conservation of the candidate peptide hormones. Phylogenetic analysis was carried out using a routine in ClustalW. The expression of the candidate genes was confirmed by searching for EST databases in human and other species. The consensus secondary structure of the candidate peptides was predicted using the Network Protein Sequence Analysis server.

Expression profiling Expression patterns of cosmedin and cospeptin transcripts in human tissues were determined by high-stringency PCR, at annealing temperatures of greater than 68° C., using Human Marathon-ready cDNA libraries obtained from Clontech (Palo Alto, Calif.). For the analysis of mRNAs in the human digestive system, normalized first-strand cDNA preparations were obtained from Clontech. Primer sequences for cosmedin PCR analysis: (SEQ ID NO:20) forward 5′-CAACTACTAACTATTCCGGAGGCA-3′ and reverse (SEQ ID NO:21) 5′-CAGTTAAGCAGACTGTCTTCC AGG-3′. Primer sequences for cospeptin PCR analysis: forward (SEQ ID NO:22) 5′-AGCAATCTGCTTATTATTCTGTCGT-3′ and reverse (SEQ ID NO:23) 5′-CACTGGTTGCTTGTATTATAT CAC-3′. For expression analyses in mouse tissues, total RNA from diverse tissues were extracted using RNeasy Mini Kit (Qiagen, Valencia, Calif.). Primer sequences for cosmedin PCR analysis: forward (SEQ ID NO:24) 5′-AGAGCCGTAGGAAGGAGCTT-3′ and reverse (SEQ ID NO:25) 5′ -TGATGTTCC AGCCAGTGGTA-3′. Primer sequences for cospeptin PCR analysis: forward (SEQ ID NO:26) 5′-CTGGTATTGTCCAGGAAGATGTG-3′ and reverse (SEQ ID NO:27) 5′ -CACATCACAGCTTGGAAGTG ATA-3′.

Peptide synthesis. Peptides were synthesized based on the solid-phase fluorenylmethoxycarbonyl protocol by automated solid-phase synthesis in a Ranin Instruments Symphony-Multiplex peptide synthesizer according to the manufacturer's protocol, and analyzed by reverse phase HPLC with a Vydac C18 analytical column and mass spectrometry using a MALDI-TOF Voyager-DE RP Workstation.

Effects of peptides on gastric emptying activity Eight-week-old C57/BL6 male mice deprived of food for 20 h were given food pellets for 90 min before intraperitoneal injection with different hormones or saline. After treatment, mice were deprived of food again and killed 90 min later. The stomach was excised at the pylorus and cardia before weighing. Gastric emptying was calculated by comparing the stomach weight of treated mice to the stomach weight of control mice killed at the time of hormone injection. The rates of gastric emptying were calculated by the formula (wet weight of stomach in fed animals killed at 0 h minus wet weight at 2 h after treatment/wet weight of stomach in fed animals killed at 0 h)×100.

Effects of cospeptin on blood pressure and heart rate in normal hypertensive rats. Blood pressure measurements in conscious male Sprague-Dawley rats (7-9 weeks of age) were made in animals preadapted to the measurement procedure. Indirect systolic pressure was determined by a programmable NIBP system using the tail-cuff method (Columbus Instruments, Columbus, Ohio). Following attachment of the pressure transducer, rats were left undisturbed for 10 min before baseline measurements that spanned a 15-min interval. Following baseline measurements, rats were injected intraperitoneally with varying doses of hormones. Blood pressure and heart rate were monitored for 50 min at 25-sec intervals. Changes in blood pressure were calculated as the average of thirty measurements performed within each 5-min interval.

Ileum contraction assay. Female rats (˜350 g) were euthanized with CO₂ and the abdominal cavity was opened to expose the intestines. A 3-cm length of ileum was removed, cleared of any adhering tissue and longitudinal strips of ileal muscle (2×5 mm) were cut. On average, six preparations were obtained from each animal. The tissues were suspended in 20-ml organ baths containing Krebs-Henseleit solution maintained at 32° C. containing 2.5 mM Ca²⁺ and gassed continuously with 95% O₂/5% CO₂. The tissues were placed under a loading tension of 1 g. The tissue was equilibrated for 90 min after which a dose response curve of the peptide of interest was obtained at 0.5 log unit cumulative incremental concentrations from 10 nM to 10 μM.

Results

The human proteome was searched for known and hypothetical proteins with a signal peptide for secretion and without a transmembrane region. Using regular expression analysis, putative secreted proteins with typical dibasic cleavage sites flanking the presumed mature peptide region of 5-50 residues in length were identified. After sorting out known proteins, the mature peptide regions of novel proteins were used to query genomes of diverse vertebrates (mouse, rat, Fugu, and others) for the identification of candidate peptide hormones showing evolutionary conservation. Two novel genes were identified and named as cosmedin and cospeptin. The open reading frames for both cosmedin and cospeptin are supported by multiple ESTs, and their signal peptides for secretion are validated in several species. The cosmedin gene encodes three conserved peptides (A, B, and C) with 23, 15, and 6 residues, respectively (FIG. 1A). Among them, cosmedin-B is presumed to be amidated at its carboxyl terminus through the glycine residue conserved in multiple species (FIG. 1B). The cospeptin gene encodes at least four amidated peptides (A to D) with 37, 27, 21, and 7 residues, respectively (FIG. 2A). Based on the conserved glycine at the C terminal end of these peptides in avian and mammalian species (FIG. 2B), these peptides are presumed to be amidated.

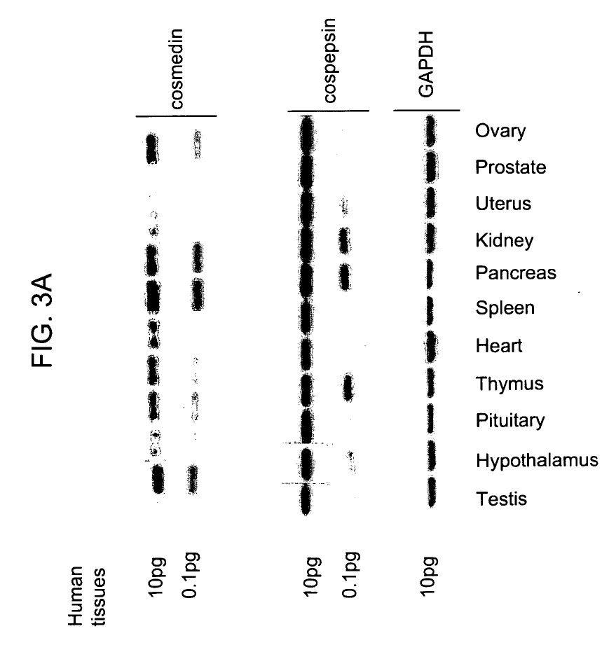

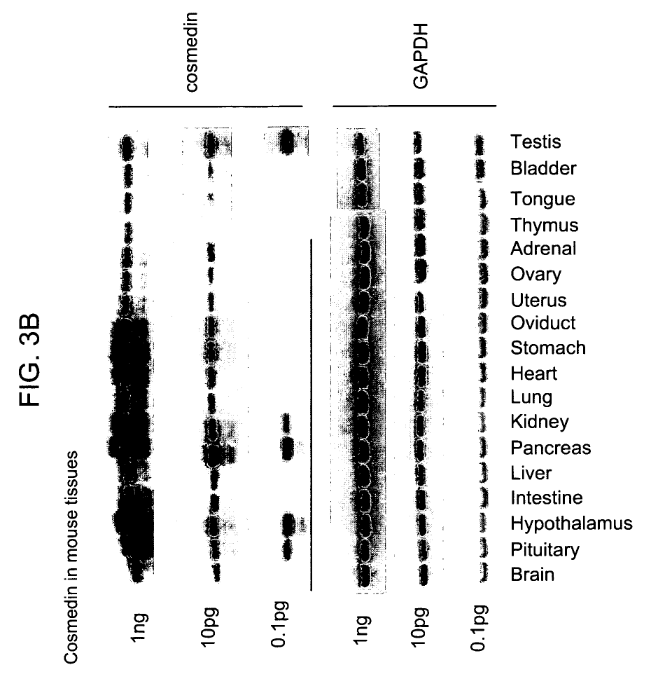

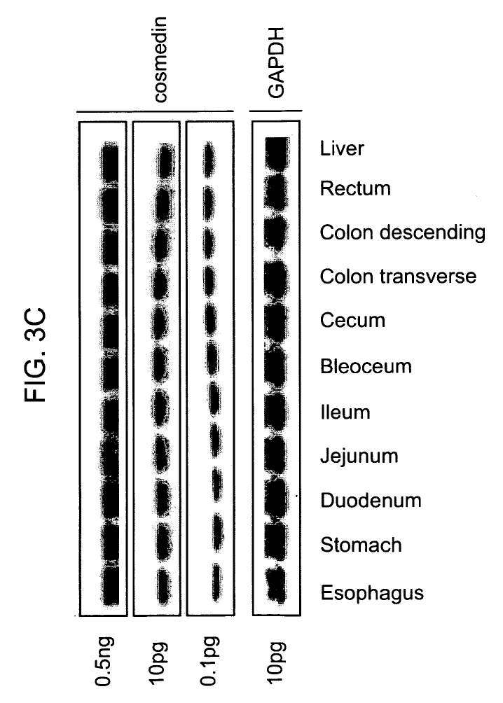

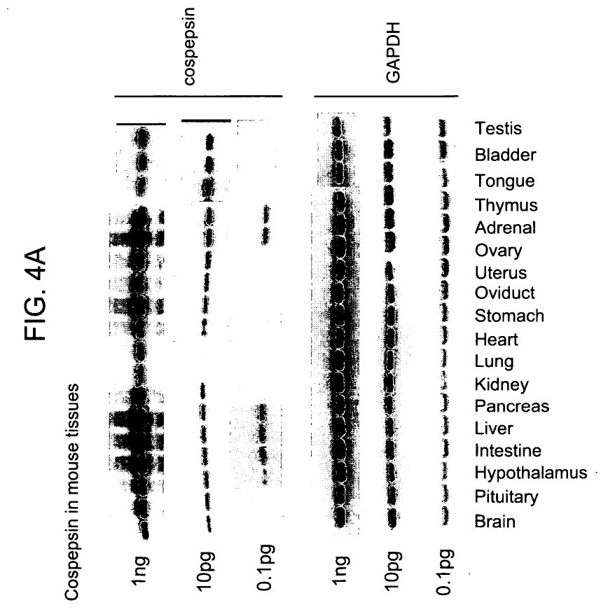

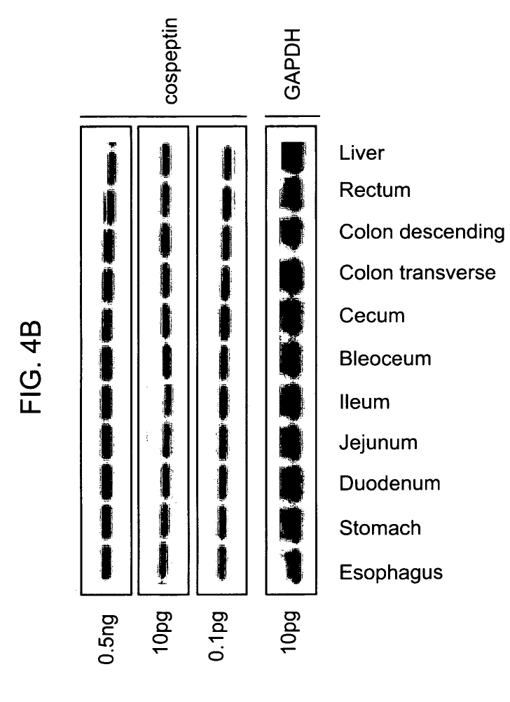

Information from the reverse transcription PCR analysis indicated that cosmedin and cospeptin genes are expressed in multiple tissues. Cosmedin transcripts are expressed in hypothalamus, pituitary, kidney, pancreas, and other tissues in both human and mouse (FIG. 3A and 3B). In addition, cosmedin is expressed in diverse human gastrointestinal tract tissues (FIG. 3C). Likewise, cospeptin is expressed in diverse human and mouse tissues, including hypothalamus, pancreas, thymus, and others (FIG. 4A). This gene is also expressed throughout the gastrointestinal tract (FIG. 4B).

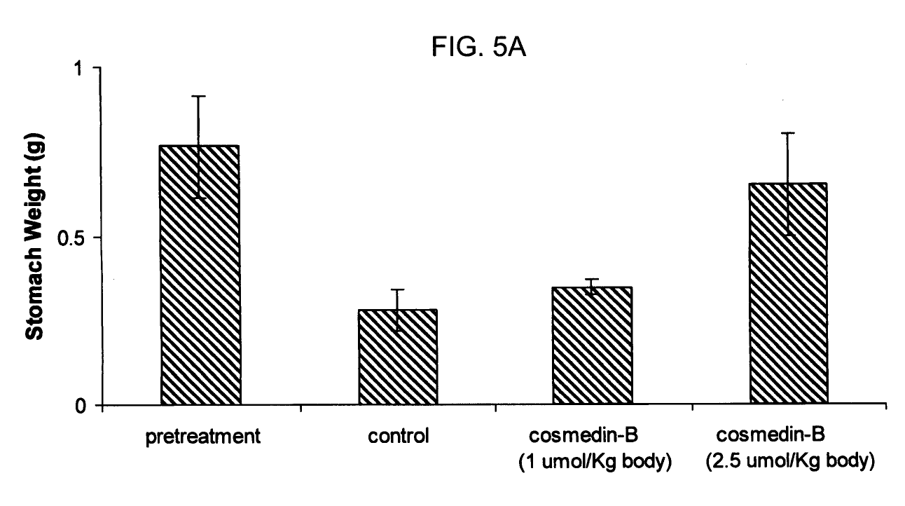

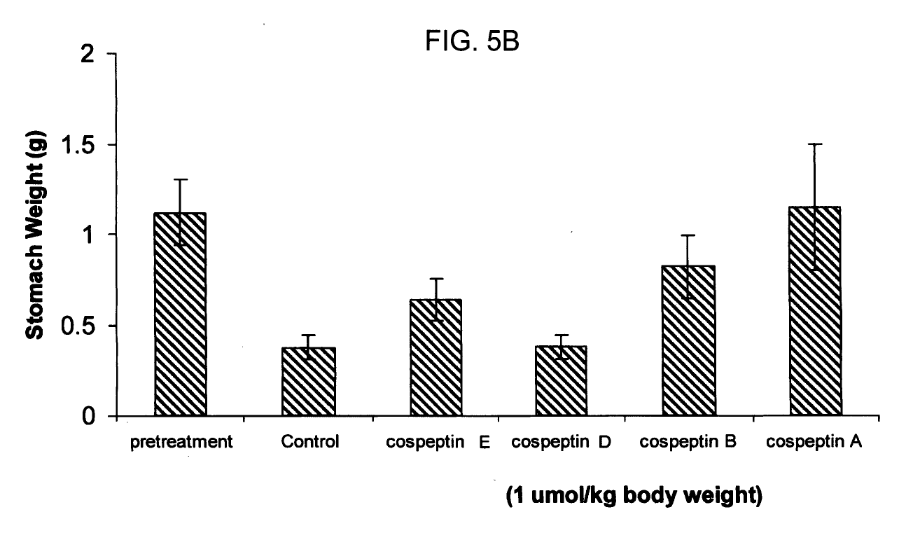

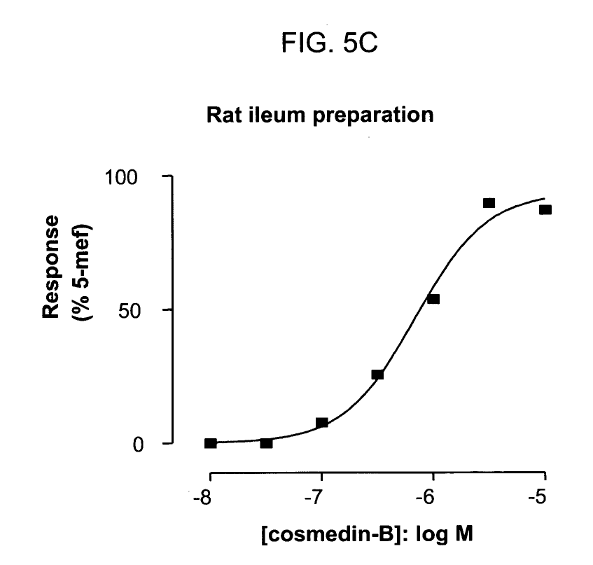

To investigate the functional roles of these conserved peptide hormones, synthetic peptides corresponding to the putative mature regions of both cosmedin and cospeptin were synthesized and used for functional analyses. As shown in FIG. 5A, intraperitoneal treatment with cosmedin-B suppressed gastric emptying activity in a dose-dependent manner. In the same bioassay, treatment with cospeptin-A, -B, and -E, also exhibited gastric emptying activities (FIG. 5B). In an organ contraction assay using rat ileal tissue strips, incubation with cosmedin-B produced a concentration-dependent muscle contraction (FIG. 5C). Results were expressed as a percentage of the maximum response to the acetylcholine muscarinic receptor agonist 5-methylfurmethide. This normalization procedure demonstrates that the magnitude of contraction induced by cosmedin-B is comparable to that mediated by the muscarinic receptor stimulation. The potency of cosmedin-B in this tissue was found to be pA₅₀=6.05±0.17.

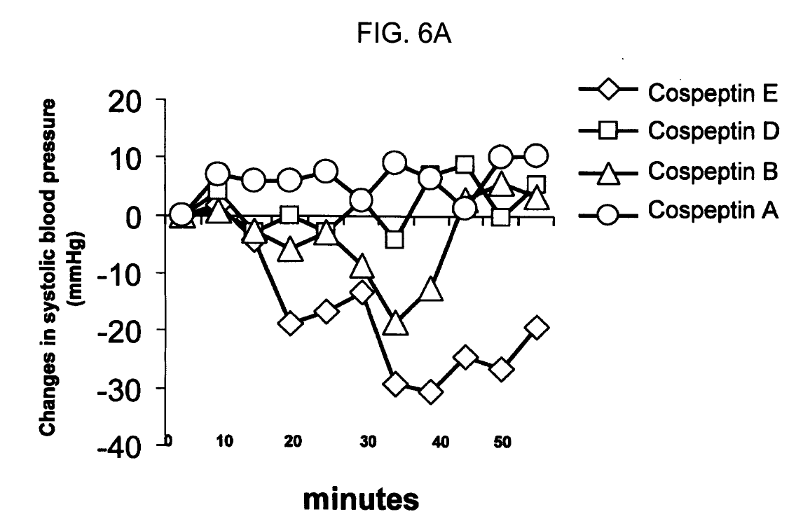

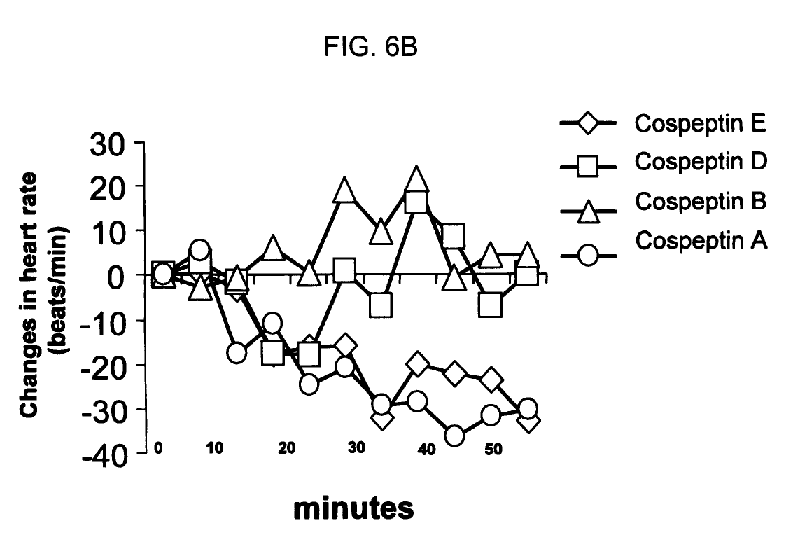

Systemic action of cospeptin on cardiovascular functions. Because cospeptin could be released into systemic circulation to act on diverse peripheral tissues, we tested the effect of cospeptin peptides on blood pressure regulation in normal rats using a noninvasive monitoring approach. As shown in the FIG. 6A, intraperitoneal administration of cospeptin B and E suppressed blood pressure in normal Sprague-Dawley rats whereas cospeptin A and D have minimal effects. In addition, treatment of cospeptin A, D, and E also decreased heart rate (FIG. 6B). Thus, cospeptin peptides are specific ligands for the signaling in the cadiovascular system and are important in the mediation of vascular responses for homeostasis.

Despite intensive effort, functional annotation has been assigned only to about one-third of the estimated 35,000 genes in the human genome. Based on sequence homology, novel paralogous ligands have been identified. However, it is difficult to identify unknown ligands with no homology to known ligands. Using Regular Expression searches for secreted proteins with conserved regions flanking dibasic residues, we have identified novel ligands for peptide hormone receptors and found them to be bioactive regulatory peptides with unique tissue expression and function.

Many fundamental biological processes involve protein-protein interactions, and comprehensively identifying them is important to systematically defining their cellular roles. Although high throughput yeast two-hybrid screening has been used to reveal protein-protein interactions, a similar approach is not generally applicable to reveal interactions between plasma membrane receptors and their extracellular ligands due to the unique topology of the membrane receptors and the ligand-receptor interactions. Based on the characteristic signatures of polypeptide hormones and the evolutionary conservation of the mature regions of candidate peptides, we were able to find peptide hormones with biological functions. Both cosmedin and cospeptin are expressed in multiple tissues with the gastrointestinal tracts as one of the major sites of expression. Using gastric emptying tests, we demonstrated biological actions of several of the predicted peptides. In addition, in vitro tests using ileal strips indicated the direct action of cosmedin-B on muscle contraction.

### EXAMPLE 2

### COSPEPTIN E BINDING

Cospeptin E was modified by the addition of a tyrosine residue at the N-terminus (zero position). The modified peptide is referred to as Tyr0-cospeptin E. The modified polypeptide retains its biological activity, but can be readily labeled by iodination. Tyr0-cospeptin was labeled with I¹²⁵ and used to detect high affinity, hormone-specific binding to different tissues.

Methods:

Radiolabeling of cospeptin. Peptide iodination was performed according to the IODO-GEN procedure (Pierce, Upland, Ind.). The mixture of Tyr0-cospeptin E (20 μg) and 1 mCi [¹²⁵I] Nal was transferred to precoated IODO-GEN vials before incubation at room temperature for 4 min. The ¹²⁵I-labeled peptide was applied to a Sep-Pak C18 cartridge (Waters, Milford, Mass.) for purification and was eluted with 60% acetonitrile/0.1%TFA. The purified tracer was stored at −20° C.

Receptor binding experiments. Homogenates were prepared from different tissues of adult rats. Binding assays were performed in glass tubes. Routinely, tissue homogenates were incubated in 100 μl of binding buffer (PBS with 0.1% bovine serum albumin) for 16 h at room temperature with varying concentrations of ¹²⁵I-Tyr0-cospeptin E (100, 000 cpm˜2 nM) in the presence or absence of unlabelled Tyr0-cospeptin E (in 5,000 fold excess). After incubation, the tubes were centrifuged for 10 min at 10,000×g, and the pellet was washed twice in ice-cold PBS. Finally, Tyr0-cospeptin E bound to the tissues was counted using a gamma-spectrophotometer. Specifically bound counts were calculated by subtracting unspecific counts determined in the presence of 5,000-fold excess of nonlabeled peptide from total counts bound to the tissues. For displacement ¹²⁵I-Tyr0-cospeptin E binding, increasing concentrations of Tyr0-cospeptin E or other peptides or analogues (cospeptin E, cospeptin A, cospeptin B, cospeptin D, cospeptin F or cosmedin B) were added.

Results

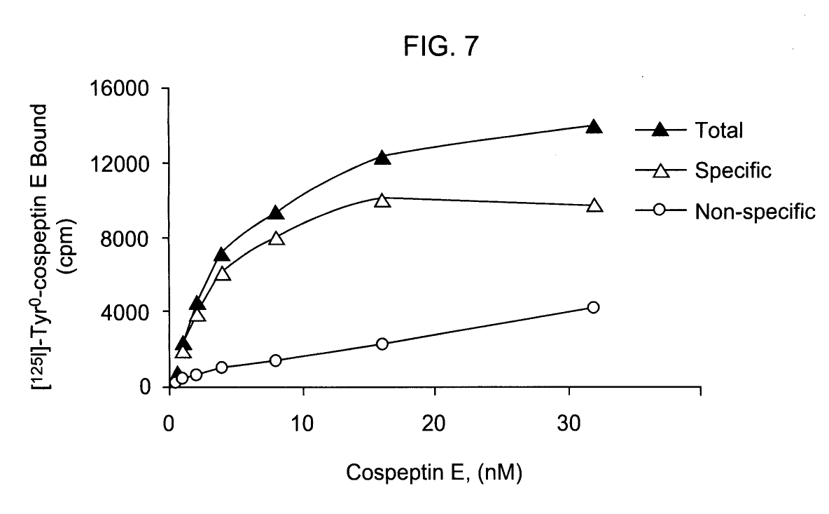

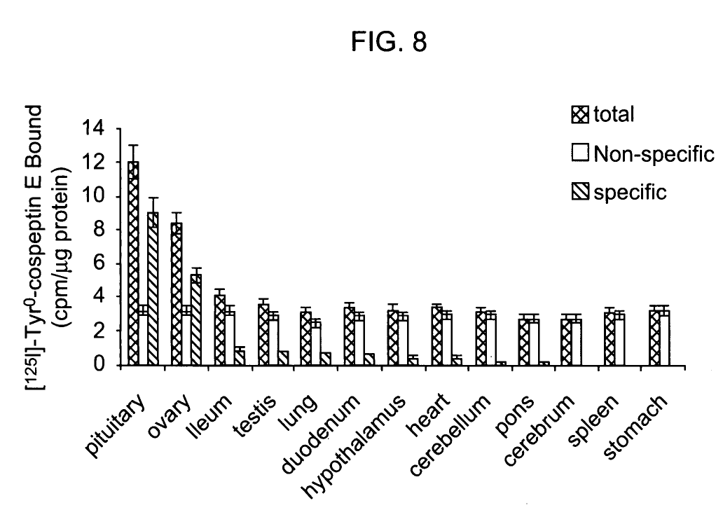

As shown in FIG. 7, a saturation curve of cospeptin E binding to pituitary glands gave a Kd value of 5 nM, indicating the presence of a specific cospeptin E receptor in pituitary tissues. High binding was also found for cospeptin in the ovaries; with lower binding in testis, lung, duodenum, hypothalamus, and heart (see FIG. 8).

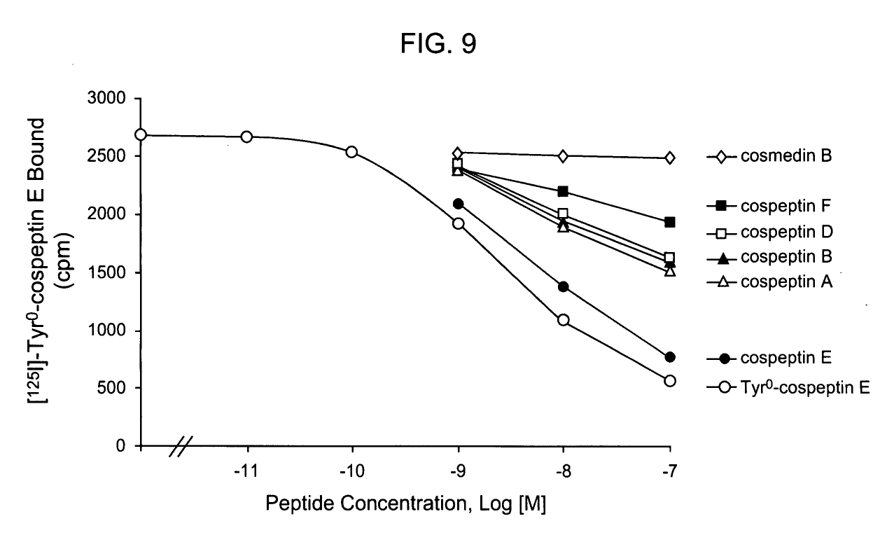

The specificity of cospeptin binding in pituitary is shown in FIG. 9. Competition studies indicated the following order of affinity to the cospeptin receptors: cospeptin E=Tyr0-cospeptin E>cospeptin A, B>>cospeptin F>>cosmedin.

1. An isolated polypeptide encoded by a nucleic acid molecule comprising a nucleotide sequence selected from the group consisting of: SEQ ID NO:1; SEQ ID NO:2; a sequence that hybridizes under stringent conditions to SEQ ID NO:1 or SEQ ID NO:2; and functional fragments, derivatives and homologs thereof.

2. An isolated polypeptide according to claim 1, wherein said polypeptide is a protein having an amino acid sequence selected from those set forth in FIGS. 1A and 1B.

3. An isolated polypeptide according to claim 1 that is a cosmedin A, B, or C peptide having a sequence selected from those set forth in FIG. 1B.

4. An isolated polypeptide according to claim 3, wherein said peptide comprises the amino acid sequence set forth in any one of SEQ ID NO:3, 4, 5, or a homolog thereof

5. An isolated polypeptide according to claim 1 that is a cospeptin A, B, C, D, E, or F peptide having a sequence selected from those set forth in FIG. 2B.

6. An isolated polypeptide according to claim 5, wherein said peptide comprises the amino acid sequence set forth in any one of SEQ ID NO:6, 7, 8, 9, 10 and 11, or a homolog thereof.

7. An isolated polypeptide comprising at least 6 contiguous amino acids of the sequence set forth in FIG. 1B or FIG. 2B.

8. A vector comprising an isolated nucleic acid molecule as defined in claim 1 operably linked with a promoter sequence.

9. A host cell transformed with the vector of claim 8.

10. A pharmaceutical composition comprising: a therapeutically effective amount of a cosmedin peptide or cospeptin peptide comprising at least 6 contiguous amino acids of an amino acid sequence set forth in FIG. 1B or FIG. 2B; and a pharmaceutically acceptable carrier.

11. A method of modulating gastrointestinal motility, the method comprising administering to an individual a therapeutically effective amount of a cospeptin or cosmedin peptide comprising at least 6 contiguous amino acids of an amino acid sequence set forth in FIG. 1B or FIG. 2B.

12. The method according to claim 11, wherein said administering is performed prior to a gastrointestinal diagnostic procedure.

13. The method according to claim 11, wherein said individual suffers from a gastrointestinal disorder.

14. The method according to claim 11, wherein said gastrointestinal disorder is a spasm.

15. The method according to claim 12, wherein said gastrointestinal disorder is a post-prandial dumping syndrome.

16. The method according to claim 12, wherein said gastrointestinal disorder is a post-prandial hyperglycemia.

17. A method of modulating gastric emptying, the method comprising administering to an individual an antagonist of cosmedin or cospeptin.

18. The method according to claim 17, wherein said individual suffers from diabetic neuropathy.

19. The method according to claim 17, wherein said individual suffers from anorexia nervosa.

20. A method of modulating hypertension, the method comprising administering to an individual a therapeutically effective amount of a cospeptin or cosmedin peptide comprising at least 6 contiguous amino acids of an amino acid sequence set forth in FIG. 1B or FIG. 2B.

21. An antibody that specifically recognizes a cospeptin or cosmedin peptide.

22. A model for cospeptin or cosmedin gene function, comprising a transgenic cell or non-human animal comprising an introduced alteration in a cospeptin or cosmedin gene.

23. A method of screening compounds to identify biologically active agents that modulate cospeptin or cosmedin function, the method comprising:
combining a compound with:
(a) a mammalian cospeptin or cosmedin peptide;
(b) a cell comprising a nucleic acid encoding a mammalian cospeptin or cosmedin peptide; or
(c) a non-human transgenic animal model for cospeptin or cosmedin gene function comprising (i) a knockout of an cospeptin or cosmedin gene or (ii) an exogenous and stably transmitted mammalian cospeptin or cosmedin gene sequence; and
determining the effect of said compound on cospeptin or cosmedin function.


SEQUENCE LISTING (27 sequences)

SEQ ID NO: 1 - DNA, 890 bp, H. sapien
aaaactcacagataagttatagttatttcaggttctgaaaagacgcagaacatgaagggactcagaagtctggcagcaacaaccttggctcttttcctggtgtttgttttcctgggaaactccagctgcgctccgcagagactgttggagagaaggaactggactcctcaagctatgctctacctgaaaggggcacagggtcgccgcttcatctccgaccagagccggagaaaggacctctccgaccggccactgccggaaagacgaagcccaaatccccaactactaactattccggaggcagcaaccatcttactggcgtcccttcagaaatcaccagaagatgaagaaaaaaactttgatcaaaccagattcctggaagacagtctgcttaactggtgaaaatatactggattatgtttaattatggttctattctctttgaaaacatgaaccatgtgaataaaacctttggacccttttattccatttgtaatcttaagaacacacacagatagtttattctttcagaaataaaatatatataggatgcttagctgagaacatcatcttctttcatgtgcttcaggtcctgtttagatgaccaaaaagtgtcttcagatcaccttgtgtcttactcttgagtttcttagaatatttataacttataaggctgaagactaaagtgttctttccttttaactatagccagtacctgtcttgatcttagtgtgtcaaaggacccatttgtgttacccacttgcatttggtgttcactccagcagaaattctccttctctggttaccttttatcccatcccaagaatggggacggaaggtgacaaaactggcggatgggacatcggagaaggatgacccccaatgtcatc

SEQ ID NO: 2 - protein, 116 aa, H. sapien
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 3 - DNA, 737 bp, H. sapien
agaactagtggatccccctttctgcaggaattcggcacgaggcggggtcattgcccggccgtcggtaacctggtttccgagagtgccgggcggtcggcgggtcagggcagcccggggcctgacgccatgtcccggaacctgcgcaccgcgctcattttcggcggcttcatctccctgatcggcgccgccttctatcccatctacttccggcccctaatgagattggaggagtacaagaaggaacaagctataaatcgggctggaattgttcaagaggatgtgcagccaccagggttaaaagtgtggtctgatccatttggcaggaaatgagagggctgtcatcagctctgattaagaaaggagatttcttcatgctttcgattctgcatggggtacagccagtcacctcaccagagaatgacggctggagaagaaaactctgtagtaccataaataagagtgcttgtaataaaagactgtgcacaaggattaatatttcccttcttaagtatcaaaagaactctggaacaaattataccattaggaaggttttcatgattcagttgatcttccaaaaatgaagctatctcacccagctgggcttggaggagcaatctggctattattctgtcggtaccacttactcaagcgagctgtgatatgaattcaagccacccagtggcttcgggaaggtccgggtctcttcttgcatctttcatataagaagatttcaataa

SEQ ID NO: 4 - protein, 67 aa, H. sapien
MSRNLRTALIFGGFISLIGAAFYPIYFRPLMRLEEYKKEQAINRAGIVQEDVQPPGLKVWSDPFGRK

SEQ ID NO: 5 - protein, 28 aa, H. sapien
RRNWTPQAMLYLKGAQGRRFISDQSRRK

SEQ ID NO: 6 - protein, 28 aa, Rattus norvegicus
KRNWTPQAMLYLKGAQGHRFISDQSRRK

SEQ ID NO: 7 - protein, 28 aa, Mus musculus
KRNWTPQAMLYLKGAQGRRFLSDQSRRK

SEQ ID NO: 8 - protein, 27 aa, Bos taurus
RRNWTPQAMLYLKGAQGRRFLSDQSRR

SEQ ID NO: 9 - protein, 28 aa, Xenopus laeris
RRNWTPQAMLYLKGAQGRRFISDESQRK

SEQ ID NO: 10 - protein, 46 aa, H. sapien
FYPIYFRPLMRLEEYKKEQAINRAGIVQEDVQPPGLKVWSDPFGRK

SEQ ID NO: 11 - protein, 46 aa, rat
FYPIYFRPLLRLEEYQKEQAVNRAGIVQEDVQPPGLKVWSDPFGRK

SEQ ID NO: 12 - protein, 46 aa, mouse
FYPIYFRPLMRLEEYQKEQAVNRAGIVQEDVQPPGLKVWSDPFGRK

SEQ ID NO: 13 - protein, 46 aa, bovine; identical to SEQ ID NO: 10

SEQ ID NO: 14 - protein, 46 aa, pig
FYPIYFRPLMRLEEYQKEQAINRAGVVQEDVQPPGLKVWSDPFGRK

SEQ ID NO: 15 - protein, 46 aa, gallus
FYPIYFRPLLLPEEYKREQSINRAGIVQENIQPPGLKVWSDPFGRK

SEQ ID NO: 16 - protein, 46 aa, xenopus
FYPIFFHPLMHIDDYKKEQATNRTDVIQEDVQPPGLKVWSDPFSRK

SEQ ID NO: 17 - protein, 46 aa, silurana
FYPIFFHPLMHIDEYKKEQAVNRTDVIQENVQPTGLKVWSDPFSRK

SEQ ID NO: 18 - protein, 46 aa, zebrafish
FYPIFFHPLTHSEDYKQVQKVNRAQVNQADIQPVGVKVWSDPYKPK

SEQ ID NO: 19 - protein, 46 aa, fugu
FYPIFFYPYMHKREYSKVQKNNRDGINQADVQPVGVKVWSDPFKPK

SEQ ID NO: 20 - DNA, 24 bp, H. sapien
caactactaactattccggaggca

SEQ ID NO: 21 - DNA, 24 bp, H. sapien
cagttaagcagactgtcttccagg

SEQ ID NO: 22 - DNA, 25 bp, H. sapien
agcaatctgcttattattctgtcgt

SEQ ID NO: 23 - DNA, 24 bp, H. sapien
cactggttgcttgtattatatcac

SEQ ID NO: 24 - DNA, 20 bp, H. sapien
agagccgtaggaaggagctt

SEQ ID NO: 25 - DNA, 20 bp, H. sapien
tgatgttccagccagtggta

SEQ ID NO: 26 - DNA, 23 bp, H. sapien
ctggtattgtccaggaagatgtg

SEQ ID NO: 27 - DNA, 23 bp, H. sapien
cacatcacagcttggaagtgata